(19) United States
(12) Patent Application Publication
(10) Pub. No.: US 2021/0095269 A1
(43) Pub. Date: Apr. 1, 2021

(54) Method For Isolating DNA By Using Cas Protein System

(71) Applicant: Cure Genetics Co., Ltd., Suzhou (CN)

(72) Inventors: Yao Wu, Suzhou (CN); Xingxing Wang, Suzhou (CN); Qiushi Li, Souzhou (CN)

(21) Appl. No.: 16/960,037

(22) PCT Filed: Dec. 30, 2018
(86) PCT No.: PCT/CN2018/125973

§ 371 Date: Jul. 3, 2020

(30) Foreign Application Priority Data
Jan. 3, 2018 (CN) 201810001073.0

(51) Int. Cl. C12N 15/10; C12Q 1/6806; C12N 15/11; C12N 9/22

(52) U.S. Cl. CPC C12N 15/1006; C12N 9/22; C12N 15/11; C12Q 1/6806

(57) ABSTRACT

The present invention relates to a method for isolating DNA from a solution, and relates in particular to a method and kit for isolating DNA from a solution by using a Cas protein. The present invention may effectively increase efficiency in extracting cell-free DNA.

### TECHNICAL FIELD

The present invention relates to a method for isolating DNA, and relates in particular to a method for isolating DNA from a solution by using a Cas protein.

### BACKGROUND ART

Under normal circumstances, the vast majority of deoxyribonucleic acids (DNA) are located in cells. However, fragments of DNA are often found outside cells, regardless of whether they are cells of microorganisms, plants or animals. Such nucleic acids are collectively referred to as cell-free DNA. The discovery of cell-free DNA in peripheral blood was reported, for the first time, by Mandel and Métais in 1948 (Mandel P., C R Hebd Seances Acad Sci (Paris), 1948, 142 (3): 241-3). After half a century of research, scientists have found that both healthy and diseased populations have a small portion of DNA which is free of cells. Such DNA is usually found in blood, plasma, serum or other body fluids. Extracellular DNA found in various samples is not easily degraded because it is protected by nucleases (for example, they are secreted in the form of protein-lipid complexes, often bound to proteins, or encapsulated in vesicles). The content of such extracellular DNA, in populations having diseases such as malignant tumors and infectious diseases, is often much higher than that in normal people, and therefore, has great application prospects in disease screening, diagnosis, prognosis, disease progression monitoring, and even in the identification of therapeutic targets. In addition, maternal blood is often accompanied by a considerable amount of fetal DNA, which can be used for a variety of detections, such as gender identification, test and evaluation of chromosomal abnormality, and can also be used for monitoring pregnancy-related complications. Just because of the strong clinical diagnostic relevance, cell-free DNA has a huge application potential in noninvasive diagnosis and prognosis, and can be used in, for example, noninvasive prenatal genetic detection, tumor detection, transplantation medicine and the detections of many other diseases.

At present, many methods for extracting and analyzing single-stranded or double-stranded cell-free DNA from various biological fluids have been reported, including an anion exchange method (WO 2016198571 A1), a silica bead extraction method (WO 2015120445 A1), and a Triton/heating/phenol method (Xue et al. Clinica Chimica Acta 2009. 404:100-104), etc. These methods usually use a chaotropic agent to release the cell-free DNA from the encapsulation of proteins, and then isolate the cell-free DNA by means of physical adsorption or precipitation. However, the yield of DNA extracted by using these methods is very low and it is difficult to extract small amounts of DNA from large samples. For example, at present, the commercially available QIAamp® serum/plasma nucleic acid purification kit (Circulating Nucleic Acid kit, QIAGEN Inc.) for cell-free nucleic acid extraction has a cell-free DNA extraction efficiency of less than 50%.

The clustered regularly interspaced short palindromic repeats (CRISPR) and CRISPR binding protein (Cas protein) form a set of a powerful nuclease system capable of cleaving almost all genomic sequences adjacent to the protospacer-adjacent motif (PAM) in eukaryotic cells (Cong et al. Science 2013. 339: 819-823). So far, all applications related to the CRISPR/Cas system further comprise, in addition to the Cas protein, an RNA component. The RNA component is a double-stranded guide RNA structure composed of a CRISPR RNA (crRNA) and a trans-activated crRNA (tracrRNA), which directs the Cas protein to cleave DNA sites complementary to the crRNA sequence (Jinek et al. Science 2012. 337: 816-821). In order to further simplify the CRISPR/Cas9 system, researchers have used engineering methods to join parts of the two components of the double-stranded guide RNA (crRNA and tracrRNA) into a chimeric single-stranded guide RNA (sgRNA). This embodies the powerful gene recognition and gene editing capabilities of the CRISPR/Cas9 system, however, the nature of the Cas protein itself has not been studied thoroughly. So far, no researchers have discovered the potential of the Cas protein and a variant thereof as a nuclease alone for recognizing and binding to DNA.

At present, there is a strong demand on the market for an efficient method for extracting cell-free DNA in order to efficiently extract the cell-free DNA from large samples of various biological fluids for subsequent analysis and detection.

### SUMMARY OF THE INVENTION

In order to improve the extraction efficiency of DNA in a solution, the present invention provides the following technical solutions:

(1) A method for isolating DNA from a solution by using a Cas protein.

(2) The method according to technical solution (1), comprising the steps of a) mixing a DNA solution with a Cas protein system; and c) isolating DNA from a complex formed by the binding of the Cas protein system with DNA.

(3) The method according to technical solution (1), comprising the steps of a) adding the Cas protein system to the DNA solution; b) enriching the complex formed by the binding of the Cas protein system with DNA in step a); and c) isolating DNA from the enriched complex in step b).

(4) The method according to technical solution (2), wherein the Cas protein system is a Cas protein coupled with a plate-like, film-like or columnar solid support.

(5) The method according to technical solution (3), wherein the Cas protein system is a Cas protein coupled with a bead-shaped solid support.

(6) The method according to technical solution (3), wherein the Cas protein system is a Cas fusion protein formed by the Cas protein and at least one linker sequence.

(7) The method according to technical solution (6), wherein in step b), a solid support coupled with an affinity molecule is added to the solution, then the enrichment is performed by centrifugation or magnetism.

(8) The method according to technical solution (6), wherein a solid support coupled with an affinity molecule is added in step a), then the enrichment is performed, after incubation, in step b).

(9) The method according to any one of technical solutions (1)-(8), wherein the DNA is double-stranded DNA.

(10) The method according to technical solution (4) or (5), wherein the Cas protein system is formed by coupling the Cas protein to a solid support through a chemical covalent bond.

(11) The method according to technical solution (6), wherein the Cas protein system is formed by combining the Cas fusion protein with the affinity molecule on the solid support.

(12) The method according to any one of technical solutions (4)-(11), wherein the solid support is a solid substance capable of forming a chemical bond with an amino acid residue.

(13) The method according to technical solution (10), wherein the solid support is selected from at least one of solid substances made of gel materials, magnetic materials, cellulose materials, silica gel materials, glass materials and artificial high-molecular polymers.

(14) The method according to any one of technical solutions (5)-(13), wherein the solid support is a magnetic bead, gel bead, and/or silica gel bead.

(15) The method according to any one of technical solutions (6)-(14), wherein the linker sequence and the affinity molecule are selected from one of the following combinations of ligand and receptor: enzyme and substrate, antigen and antibody, and biotin and avidin.

(16) The method according to technical solution (15), wherein the enzyme and substrate are selected from glutathione transferase and glutathione.

(17) The method according to technical solution (15), wherein the antigen and antibody are selected from the group of: a protein A or a fragment thereof which retains the Fc region-binding function and an immunoglobulin or Fc or Fab fragments thereof, a protein G or a fragment thereof which retains the Fc region-binding function and an immunoglobulin or Fc or Fab fragments thereof, a histidine tag and an anti-histidine tag, a polyhistidine tag and an anti-polyhistidine tag, and a FLAG tag and an anti-FLAG tag.

(18) The method according to technical solution (15), wherein the biotin and avidin are selected from the group of: biotin and avidin or streptavidin, biotin-linked biotin receptor protein and avidin or streptavidin, Strep-tag and avidin or streptavidin.

(19) The method according to any one of technical solutions (2)-(18), wherein the reaction time of step a) is from 5 minutes to 6 hours, preferably the reaction time is from 15 minutes to 1 hour.

(20) The method according to any one of technical solutions (2)-(18), wherein the reaction temperature of step a) is 20° C.-37° C., preferably the reaction temperature is 30° C.-37° C.

(21) The method according to any one of technical solutions (7) and (12)-(20), wherein in step b), the reaction time of adding a solid support coupled with an affinity molecule to the solution is from 30 minutes to overnight, preferably the reaction time is 1-2 hours.

(22) The method according to technical solution (21), wherein the reaction temperature of step b) is 4-37° C., preferably the reaction temperature is 20-30° C.

(23) The method according to any one of technical solutions (3)-(22), wherein the enrichment of step b) is performed by centrifugation or magnetism.

(24) The method according to any one of technical solutions (2)-(23), wherein in step c), DNA is isolated by elution.

(25) The method according to any one of technical solutions (2)-(24), wherein the Cas is a Cas protein with a DNA binding function.

(26) The method according to technical solution (25), wherein the Cas protein is a natural Cas protein, a mutant Cas protein which loses the gRNA binding ability, or a mutant Cas protein which loses the nuclease activity.

(27) The method according to technical solution (25) or (26), wherein the Cas protein is a Cas9 protein.

(28) The method according to technical solution (27), wherein the Cas9 protein is a dCas9 protein.

(29) A kit for isolating DNA from a solution, comprising components a) a Cas protein system, and b) a DNA eluent.

(30) The kit according to technical solution (28), wherein component a) the Cas protein system is a solution system containing a Cas protein coupled with a solid support, or is a solution system containing a Cas fusion protein and a solid support coupled with an affinity molecule.

(31) The kit according to technical solution (29), wherein component a) the Cas protein system is composed of two independent systems: i) a Cas fusion protein formed by the Cas protein and at least one linker sequence, and ii) a solid support coupled with an affinity molecule.

(32) The kit according to technical solution (29), wherein component b) the DNA eluent may be the following liquid: 0.5-10 mg/ml proteinase K solution, 1-5 M concentration of NaCl or KCl saline solution, alkaline eluent at pH 11-12.5 with 0-0.3 M Tris and 0-0.5 M NaCl.

(33) The kit according to technical solution (30) or (31), wherein the solid support is a plate-like, film-like or columnar solid support.

(34) The kit according to technical solution (33), wherein the solid support is selected from at least one of solid substances made of gel materials, magnetic materials, cellulose materials, silica gel materials, glass materials and artificial high-molecular polymers.

(35) The kit according to technical solution (34), wherein the solid support is a magnetic bead, gel bead, and/or silica gel bead.

(36) The kit according to any one of technical solutions (31)-(35), wherein the linker sequence and the affinity molecule are selected from one of the following combinations of ligand and receptor: enzyme and substrate, antigen and antibody, and biotin and avidin.

(37) The kit according to technical solution (36), wherein the and substrate are selected from glutathione transferase and glutathione.

(38) The kit according to technical solution (37), wherein the antigen and antibody are selected from the group of: a protein A or a fragment thereof which retains the Fc region-binding function and an immunoglobulin or Fc or Fab fragments thereof, a protein G or a fragment thereof which retains the Fc region-binding function and an immunoglobulin or Fc or Fab fragments thereof, a histidine tag and an anti-histidine tag, a polyhistidine tag and an anti-polyhistidine tag, and a FLAG tag and an anti-FLAG tag.

(39) The kit according to technical solution (36), wherein the biotin and avidin are selected from the group of: biotin and avidin or streptavidin, biotin-linked biotin receptor protein and avidin or streptavidin, Strep-tag and avidin or streptavidin.

(40) The kit according to any one of technical solutions (29)-(39), wherein the Cas is a Cas protein with a DNA binding function.

(41) The kit according to technical solution (40), wherein the Cas protein is a natural Cas protein, a mutant Cas protein which loses the gRNA binding ability, or a mutant Cas protein which loses the nuclease activity.

(42) The kit according to technical solution (41), wherein the Cas protein is a Cas9 protein.

(43) The kit according to technical solution (42), wherein the Cas9 protein is a dCas9 protein (a mutant Cas9 protein which loses nuclease activity).

Under the condition of the same content of DNA samples, the extraction method of the present invention eliminates cumbersome sample processing processes, reduces requirements for sample usage amounts, and greatly increases the efficiency of extracting cell-free DNA.

### BRIEF DESCRIPTION OF THE DRAWINGS

FIG. 1: expression vector pSMART-his-S.P.dCas9-FLAG containing the his-S.P.dCas9-FLAG gene fragment.

FIG. 2: electrophoresis band of cell-free DNA (SEQ ID NO. 8) of 486 bp in length bound to a dCas protein.

Lane 1 is the band of cell-free DNA itself.

Lane 2 shows the situation after the dCas9 and cell-free DNA (SEQ ID NO. 8) are bound.

### DETAILED DESCRIPTION OF THE INVENTION

### Cas Protein

Clustered regularly interspaced short palindromic repeats (CRISPR) and the CRISPR binding protein (Cas protein) form a powerful nuclease system. The CAS protein can be divided into four different functional modules: a target recognition module (interval acquisition); an expression module (crRNA processing and target combination); an interference module (target cleaving); and an auxiliary module (supervision and other CRISPR-related functions). In recent years, a large amount of structural and functional information has been accumulated for core Cas proteins (Cas1-Cas10), which allows them to be classified into these modules. The CRISPR-related endonuclease Cas protein can target specific genomic sequences through the single-stranded guide RNA (sgRNA). Taking the Streptococcus pyogenes Cas9 protein (spCas9) as an example, the protein comprises a double-leaf structure composed of a target recognition module and a nuclease module (interference module), which accommodates the sgRNA-DNA heteroduplex in the positively-charged groove at the interface. Moreover, the target recognition region is necessary for binding sgRNA and DNA. The nuclease region comprises HNH and RuvC nuclease domains, which are suitable for cleaving the complementary and non-complementary strands of the target DNA, respectively. The nuclease region also contains a carboxyl-terminal domain responsible for interaction with the protospacer-adjacent motif (PAM). However, to date, there is no report from any research group about the structural region where the Cas protein directly binds to DNA, or the crystal structure of the Cas-DNA complex.

Therefore, in the present invention, in the absence of RNA mediation, the Cas protein itself is a nucleic acid binding system. Under normal circumstances, the Cas protein can recognize and bind to almost all DNA sequences without RNA mediation, and does not cleave the bound DNA sequences.

The Cas protein of the present invention is a Cas protein with the DNA binding function, such as a natural Cas protein, a mutant Cas protein, a mutant Cas protein which loses the gRNA binding ability, and a mutant Cas protein which loses the nuclease activity (dead Cas, dCas).

Non-limiting examples of Cas proteins include: Cas1, Cas1B, Cas2, Cas3, Cas4, Cas5, Cas6, Cas7, Cas8, Cas9 (also known as Csn1 or Csx12), Cas10, Cas12, Cas13, Cas14, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and homologous proteins thereof in different species, endonuclease-inactivated mutant proteins or modified forms thereof.

In a specific embodiment, the Cas protein of the present invention is a Cas9 protein, more preferably a mutant Cas9 protein which loses nuclease activity (dead Cas9, dCas9)

Cas9, also known as Csn1 or Csx12, is a giant protein that participates in both crRNA biosynthesis and destruction of invading DNA. Cas9 has been described in different bacterial species such as S. thermophiles, Listeria innocua (Gasiunas, Barrangou et al. 2012; Jinek, Chylinski et al. 2012) and S. Pyogenes (Deltcheva, Chylinski et al. 2011). Cas9 of the present invention includes, but is not limited to, the following types: Streptococcus pyogenes Cas9 protein, and its amino acid sequence is under SwissProt database accession number Q99ZW2; Neisseria meningitides Cas9 protein, and its amino acid sequence is under UniProt database number A1IQ68; Streptococcus thermophilus Cas9 protein, and its amino acid sequence is under UniProt database number Q03LF7; and Staphylococcus aureus Cas9 protein, and its amino acid sequence is under UniProt database number J7RUA5.

The Cas protein that acts on the double-stranded DNA generally has two active sites for endonuclease. If there is and only one site is mutated or deleted to cause the enzyme activity at this site to be inactivated, a nickase that cleaves a single strand of DNA can be obtained, such as Cas9-nickase is widely used because of its high fidelity and the characteristics of cleaving a single strand of DNA.

In addition, screening for the Cas protein variant with one or more point mutations by mutation has been proved to improve its specificity, reduce the off-target rate of genome editing, or make it compatible with more diverse PAM sequences, such as eSpCas9, SpCas9-HF, HeFSpCas9, HIFI-SpCas9 and xCas9 (Christopher A. Vet al. Nature Medicine 2018 (24), pp. 1216-1224; Johnny H. H et al. Nature 2018 556 (7699), pp. 57-63).

In view of the Cas protein comprising multiple functional modules, the protein sequence is long, in order to facilitate packaging and transportation and function control, the protein sequence will also be truncated through protein engineering research to form a Cas protein system, such as a Split-SpCas9 system comprising complexes formed by truncated proteins (Jana M et al. Plant Biotechnology Journal 2017 15, pp. 917-926).

In some embodiments, the Cas protein is a fusion protein or protein complex containing the above protein or a mutant thereof, including but not limited to variants linked to other amino acid sequences on the Cas. The fusion of other functional proteins on the basis of the above Cas protein or its variants can increase the specificity and effectiveness of the Cas protein function, and can also produce effects on the genome other than cleavage. For example, the fusion of a FokI on the Cas or dCas can increase the specificity of the Cas9 protein for genomic cleavage, because only the FokI dimerization has a cleaving activity, which requires a pair of recognition regions to complete the cleavage and reduce the off-target rate. For another example, a FokI is connected to the Cas protein in which a part of the functional domains is truncated but the DNA binding ability thereof is preserved (Ma et al. ACS Synth. Biol., 2018, 7 (4), pp 978-985). For another example, a base-modifying enzyme (such as deaminase, cytosine deaminase and adenine deaminase) is fused to the Cas-nickase or dCas, which may efficiently perform targeted base modification on the target region of the genome (Komor et al. Sci. Adv. 2017.3: eaao4774). For another example, the dCas9 is fused with some protein domains that may regulate the gene expression, which may effectively regulate the expression of target genes. For example, the dCas9 fused with transcriptional activators, such as VP64, VPR and the like, may bind near the target gene to activate the expression guided by gRNA; conversely, the dCas9 fused with a transcriptional repressor (such as SRDX) will downregulate the target gene. dSpCas9-Tet1 and dSpCas9-Dnmt3a can be used to modify the epigenetic state, and regulate the methylation state of the endogenous gene promoter to regulate the protein expression.

Cas Protein with the DNA Binding Function

The Cas protein with the DNA binding function may be a natural Cas protein, or a mutation made outside the DNA-binding functional region of the Cas protein, or a Cas protein obtained after mutation in the DNA-binding functional region.

In order to detect whether the Cas protein has a DNA binding function, gel electrophoresis may be used to compare the gel band positions of the protein-bound DNA and the protein-unbound DNA for discrimination (for the detection method, see example 1).

Nuclease-Inactivated Mutant Cas Protein (dCas Protein)

The nuclease-inactivated mutant Cas protein (dCas protein) is a variant obtained by mutation of the Cas protein, and its endonuclease activity is inactivated or substantially inactivated, causing the Cas protein to lose or substantially lose the endonuclease activity, and thus fail to cleave the target sequence. The above non-limiting examples of the Cas proteins can be transformed into the dCas proteins by endonuclease-inactivating mutations, including insertion, deletion, or substitution of one or more amino acid residues.

For example, certain Cas9 mutations may cause the Cas9 protein to lose or substantially lose endonuclease activity, and thus fail to cleave the target sequence. For the Cas9 of a certain species, such as spCas9, exemplary mutations that reduce or eliminate the endonuclease activity include one or more mutations in the following positions: D10, G12, G17, E762, H840, N854, N863, H982, H983, A984, D986 or A987. The literature proves that guide RNA (gRNA)-mediated endonuclease inactivation mutation Cas9 (referred to as dCas9) can lead to the suppression of the expression of E. coli-specific endogenous genes and EGFP reporter genes in human cells (Qi et al. Cell 2013. 152:1173-1183). This study proves that the use of gRNA-mediated dCas9 technology may accurately recognize and bind to the corresponding genome.

The dCas protein may be a non-naturally occurring protein in nature, and is a variant obtained by protein engineering or by random mutagenesis, of which the endonuclease activity is inactivated or substantially inactivated. For example, the corresponding dCas9 protein can be obtained by mutation, that is, the deletion or insertion or substitution of at least one residue in the amino acid sequence of the Streptococcus pyogenes Cas9 endonuclease.

In some embodiments, the dCas9 protein may be a variant obtained by mutations inactivating or substantially inactivating the endonuclease activity of Cas9 of different species, including but not limited to, Streptococcus pyogenes Cas9 protein, Neisseria meningitides Cas9 protein, Streptococcus thermophilus Cas9 protein, Staphylococcus aureus Cas9 protein, Streptococcus pneumoniae Cas9 protein, etc.

### Linker Sequence and Affinity Molecule

The linker sequence of the present invention refers to a molecule directly or indirectly connected to the C-terminus or N-terminus of the Cas protein, which ultimately forms a fusion protein with the Cas protein.

The affinity molecule of the present invention is a molecule coupled to a solid substance and may form a specific binding with the above linker sequence on the Cas protein.

The linker sequence and the affinity molecule of the present invention have a relationship of receptor and ligand to each other. Any protein linker sequence can be used in the practice of the present invention, as long as it can specifically bind to the affinity molecule. The linker sequence and the affinity molecule are selected from one of the following combinations of ligand and receptor: enzyme and substrate, antigen and antibody, and biotin and avidin. The enzyme and substrate are selected from the group of: glutathione transferase and glutathione. The antigen and antibody are selected from the group of: a protein A or a fragment thereof which retains the Fc region-binding function and an immunoglobulin or Fc or Fab fragments thereof, a protein G or a fragment thereof which retains the Fc region-binding function and an immunoglobulin or Fc or Fab fragments thereof, a histidine tag and an anti-histidine tag, a polyhistidine tag and an anti-polyhistidine tag, and a FLAG tag (amino acid sequence: ATLAAAAL) and an anti-FLAG tag. The biotin and avidin are selected from the group of: biotin and avidin (UniProt database number P02701) or streptavidin (UniProt database number P22629), biotin-linked biotin receptor protein (such as AviTag, amino acid sequence: GLNDIFEAQKIEWHE) and avidin or streptavidin, and Strep-tag (amino acid sequence: WSHPQFEK) and avidin or streptavidin.

In the present invention, the linker sequence and the affinity molecule are specifically bound by affinity, and are interchangeable. For example, if the linker sequence is a receptor, the affinity molecule is a ligand that specifically binds to the receptor; and the linker sequence may also be a ligand, so then the affinity molecule is a receptor that specifically binds to the ligand. For another example, if the linker sequence is streptavidin, the affinity molecule is biotin; and the linker sequence may also be biotin, so then the affinity molecule is streptavidin.

### Solid Support

The solid substance of the present invention is a solid substance capable of forming a chemical bond with an amino acid residue, and thus can be coupled with an affinity molecule or a Cas protein through a chemical bond.

In terms of materials, suitable solid supports may be solid substances made of gel materials, magnetic materials, cellulose materials, silica gel materials, glass materials or artificial high-molecular polymers. The gel material may be a hydrogel, organic gel, xerogel or nano-composite hydrogel. The magnetic material may be a metal oxide (such as an oxide of iron, cobalt or nickel) wrapped by a polymer material. The cellulose material may be cellulose, cellulose acetate or nitrocellulose. The silica gel material may be a silica support or an organic silica gel support. The artificial high-molecular polymer may be nylon, polyester, polyethersulfone, polyolefin, poly(1,1-vinylidene fluoride), and combinations thereof. In terms of forms, the solid supports may be in forms of beads, chemical film-like columns, glass plates, 6-well/24-well/48-well/96-well/384-well plates, PCR tubes and the like.

After binding the DNA-bound Cas protein to a solid support, such as a chemical film-like column, glass plate, 6-well/24-well/48-well/96-well/384-well plate or PCR tube, DNA is eluted by an eluent.

After binding the DNA-bound Cas protein to a bead-shaped solid support, DNA is eluted by an eluent after enrichment, such as centrifugal enrichment or magnetic enrichment.

The solid supports of the present invention are preferably bead-shaped, such as magnetic beads, gel beads and/or silica gel beads. Non-limiting examples of the gel beads that can be used include: Anti-6X His tag® antibody (agarose gel) (Abcam), Anti-6×His AlphaLISA® Acceptor beads (PerkinElmer), Dynabeads™ His-Tag Isolation and Pulldown (ThermoFisher), Anti-His-tag mAb-Magnetic Beads (MBL Life science) and His Tag Antibody Plate (GenScript).

### Coupling

Conventional methods can be used to couple an affinity molecule or Cas protein to a solid support. For linking protein A and protein G to a solid support, see, for example, Hermanson et al. 1992, Immobilized Affinity Ligand Techniques, Academic Press. Typically, the solid support is activated with a reactive functional group (“activating group”), such as epoxide (epichlorohydrin), cyanide (cyanogen bromide, CNBr), N, N-disuccinimidyl carbonate (DSC), aldehyde or activated carboxylic acid (e.g., N-hydroxysuccinimide (NHS) ester or carbonyldiimidazole (CDI) activated ester). These activating groups can be directly attached to a solid support, such as CNBr, or they can also be part of a “linker” or spacer molecule, typically a linear chain of carbon, oxygen, and nitrogen atoms, as a ten-membered chain of carbon and oxygen found in the linker butanediol diglycidyl ether (a commonly used epoxide coupling agent). Under coupling conditions, the activated solid support is then equilibrated with the Cas protein or affinity molecule. After the coupling reaction is completed, the medium is washed thoroughly.

### Cas Fusion Protein

The Cas fusion protein of the present invention is a fusion protein formed by adding a linker sequence to the N-terminus or C-terminus of the Cas protein or any part of its protein sequence. The fusion Cas protein of the present invention can express the Cas protein and the linker sequence together by genetic engineering methods.

### Cas Protein System

The Cas protein system of the present invention is a complex containing the Cas protein, which is used to bind to DNA in a solution.

In a specific embodiment, the Cas protein system is a complex formed by coupling a Cas protein to a solid support through a chemical covalent bond.

In a specific embodiment, the Cas protein system is a Cas fusion protein formed by a Cas protein and one or more linker sequences.

In another specific embodiment, the Cas protein system is a complex formed by binding a Cas fusion protein to an affinity molecule coupled to a solid substance.

Binding of the Cas Fusion Protein to the Solid Substance Coupled with an Affinity Molecule

A Cas protein and a linker sequence together form a Cas fusion protein. The linker sequence may be directly or indirectly located at the N-terminus or C-terminus of the Cas protein. Taking the solid substance as a gel bead as an example, in a preferred embodiment, when using a gel bead coupled with an affinity molecule to bind the Cas fusion protein, for every 10 μl of gel beads, the initial concentration of Cas fusion protein in the reaction solution is 0.001-20 μg/μl, and preferably 0.01-10 μg/μl of Cas9 fusion protein before adding the gel beads.

The binding of the Cas fusion protein to the solid substance is performed at 4-37° C., and preferably 4° C. or room temperature (20-25° C.). The binding reaction solution can be deionized water, 1× phosphate buffered saline (PBS), Tris buffer (50 mM Tris-HCl, 150 mM NaCl, pH7.4), or other buffers with a salt ion concentration below 150 mM, free of oil detergents and denaturants, and with an approximately neutral pH. The reaction time is from 30 minutes to overnight, and preferably overnight.

The Cas protein may be directly bound to the solid substance coupled with an affinity molecule without a connexin.

### Binding of the Cas Protein to Cell-Free DNA

The Cas protein of the present invention may bind to any DNA in a cell-free state. The DNA may be double- or single-stranded DNA. Cell-free DNA bound to the Cas protein can be isolated from the liquid phase by binding a solid substance to the Cas protein.

DNA bound to the Cas protein of the present invention may be any cell-free DNA. For example, DNA fragment with any sequence obtained by PCR using a genome as a template, DNA fragments with sequences artificially designed, cell-free DNA after cell lysis, genome obtained after cell lysis, and cell-free DNA in human or animal body fluids. In some embodiments, DNA refers to DNA fragments obtained by PCR using a genome as a template, DNA fragments with sequence artificially designed, or cell-free DNA in human or animal body fluids.

As a preferred embodiment, when the Cas protein or Cas fusion protein binds to cell-free DNA, the protein concentration in the DNA binding reaction solution is 0.001-20 μg/μl, and preferably, 0.01-10 μg/μl of the Cas protein or a variant thereof or the Cas fusion protein. In a preferred embodiment, the reaction is performed from room temperature (20-25° C.) to 37° C. The DNA binding reaction solution can be deionized water, 1× phosphate buffered saline (PBS), Tris buffer (50 mM Tris-HC1, 150 mM NaCl, pH7.4), or other buffers with a salt ion concentration below 150 mM, free of oil detergents and denaturants, and with a neutral pH, and preferably, 50 mM KCl, 10 mM EDTA, 30 mM Tris-HCl, 0.2% Triton X-100, and 12% glycerol. The reaction time is from 5 minutes to 6 hours, and in one embodiment, the preferred time is from 5 minutes to 2 hours. In another embodiment, the preferred time is from 15 minutes to 1 hour.

Method for Isolating Cell-Free DNA from a Solution

The method for isolating cell-free DNA by using the Cas protein of the present invention may be used in three ways:

Method 1: the Cas protein or Cas fusion protein is first bound to cell-free DNA in the DNA binding reaction solution, and then in the protein binding reaction solution, the Cas protein or Cas fusion protein is bound by the solid substance coupled with an affinity molecule. Firstly, the Cas protein or Cas fusion protein and cell-free DNA are incubated and bound in the DNA binding reaction solution under the DNA binding reaction conditions described above, the preferred time is from 5 minutes to 6 hours; and in another embodiment, the preferred time is from 15 minutes to 1 hour. The reaction temperature is from room temperature (20-25° C.) to 37° C.; after the DNA binding reaction is completed, the solid substance coupled with an affinity molecule is directly added to the solution. Under the protein binding reaction conditions described above, the Cas protein or Cas fusion protein is bound to the solid substance coupled with an affinity molecule. The preferred reaction time is from 30 minutes to overnight, and the reaction temperature is from 4° C. to 37° C. Finally, the solid substance is separated by centrifugation and the supernatant is discarded. After the solid substance is washed several times, the Cas protein or a variant thereof or Cas fusion protein, and bound DNA are eluted from the solid substance to obtain an elution solution. The eluent that can be used is a 0.5-10 mg/ml proteinase K solution, 1-5 M NaCl or KCl solution, alkaline eluent (0-0.3 M Tris, 0-0.5 M NaCl, pH 11-12.5), acid eluent (0-0.3 M glycine HCl,pH 2.5-3.5), competitive eluent (DYKDDDK or FLAG amino acid in TBS, amino acid concentration can be 100 to 500 μg/ml), or PAGE gel sample solution (0.01 M Tris-HCl, 10% glycerol, 0.016% bromophenol blue).

Method 2: the solid substance coupled with an affinity molecule is first bound to the Cas protein or a variant thereof or Cas fusion protein, and then is incubated and bound to cell-free DNA in the DNA binding reaction solution. The reaction conditions, solutions and elution conditions used are the same as those in Method 1.

Method 3: the solid substance coupled with an affinity molecule, the Cas9 protein or a variant thereof, or the Cas fusion protein are simultaneously added to the DNA binding reaction solution, and the solid substance is separated after co-incubation. The solution in the method may be a solution used when the Cas protein or a variant thereof or Cas fusion protein binds to cell-free DNA, preferably the reaction time is from 30 minutes to 6 hours, and the reaction temperature is from room temperature (20-25° C.) to 37° C. The elution conditions are the same as those in Method 1.

### EXAMPLES

The examples are merely illustrative and are not intended to limit the present invention in any way.

### Example 1

### DNA Binding Activity of the Cas Protein

The DNA binding activity of the Cas protein can be determined by using gel electrophoresis to compare the gel band positions of DNA binding with or without the Cas protein. The following reaction conditions were used:

reaction buffer (BA): 50 mM tris-hydroxymethylaminomethane hydrochloride (Tris-HCl, pH 7.4); 150 mM sodium chloride (NaCl)

reaction system (20 μl):

dCas protein: 2 μg

DNA: 0.25 μg

nuclease-free double-distilled water: the volume was made up to 20 μl

reaction process:

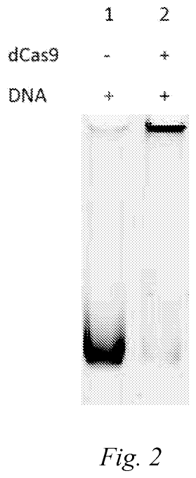

the above reaction system was added into a 200 μl PCR tube and mixed well, reacted at room temperature or 37° C. for 15 minutes to 1 hour. After the reaction was completed, a non-denaturing gel was used and the electrophoresis was run in a BioRad electrophoresis tank at 80 V and 400 mA for 80 minutes. After removing the non-denaturing gel, the nucleic acid was stained with DuRed (Shanghai Yeasen Bio Technologies Co., Ltd., 10202ES76), and then a picture (see FIG. 2) was taken by using a gel imager. The electrophoretic band of the DNA binding with Cas protein lagged behind that of the DNA binding without Cas protein significantly.

### Example 2

### Preparation of Cell-Free DNA

The cell-free DNA of the present invention can be DNA of any sequence. In this example, we synthesized and constructed 11 DNA sequences with different lengths, as shown in Table 1.

TABLE 1
Cell-free DNA sequence (5′-3′)

| SEQ ID NO: | Length (bp) |
|---|---|
| 1 | 100 |
| 2 | 100 |
| 3 | 100 |
| 4 | 200 |
| 5 | 200 |
| 6 | 200 |
| 7 | 957 |
| 8 | 486 |
| 9 | 166 |
| 10 | 609 |
| 11 | 2655 |

The DNA sequences of SEQ ID NOs. 1-6 were synthesized by PCR directly with forward and reverse primers (Table 2), without using any template. The main purpose of using these six DNA sequences was to detect whether the protospacer-adjacent motif (PAM) region would affect the binding of the Cas protein to DNA. SEQ ID NOs. 1-3 were 100 bp, SEQ ID NOs. 4-6 were 200 bp, wherein there was no PAM region (NGG) corresponding to the Cas9 in SEQ ID NO. 1 and SEQ ID NO. 4, there was one PAM region on SEQ ID NO. 2 and SEQ ID NO. 5 respectively, and there were 3 PAM regions on SEQ ID NO. 3 and SEQ ID NO. 6 respectively. Detailed results will be discussed in Example 4.

The DNA sequences of SEQ ID NOs. 7-11 were made by PCR by designing the corresponding forward and reverse primers respectively, and using the 293T cell (Shanghai Institutes for Biological Sciences, GNHu17) genome as a template.

TABLE 2
The upstream and downstream primers corresponding to different cell-
free DNA sequences, and the annealing temperature of each PCR.

| | | | Annealing |
|---|---|---|---|
| | | | temperature |
| | Upstream primer | Downstream primer | (° C.) |
| SEQ ID NO: 1 | SEQ ID NO: 12 | SEQ ID NO: 13 | 55 |
| SEQ ID NO: 2 | SEQ ID NO: 14 | SEQ ID NO: 15 | 55 |
| SEQ ID NO: 3 | SEQ ID NO: 16 | SEQ ID NO: 17 | 55 |
| SEQ ID NO: 4 | SEQ ID NO: 18 | SEQ ID NO: 19 | 58 |
| SEQ ID NO: 5 | SEQ ID NO: 20 | SEQ ID NO: 21 | 58 |
| SEQ ID NO: 6 | SEQ ID NO: 22 | SEQ ID NO: 23 | 58 |
| SEQ ID NO: 7 | SEQ ID NO: 24 | SEQ ID NO: 25 | 68 |
| SEQ ID NO: 8 | SEQ ID NO: 26 | SEQ ID NO: 27 | 60 |
| SEQ ID NO: 9 | SEQ ID NO: 28 | SEQ ID NO: 29 | 60 |
| SEQ ID NO: 10 | SEQ ID NO: 30 | SEQ ID NO: 31 | 59 |
| SEQ ID NO: 11 | SEQ ID NO: 32 | SEQ ID NO: 33 | 66 |

The PCR of each DNA sequence of SEQ ID NOs. 1-6 was performed by using two primers of upstream primer and downstream primer as described above and the Q5 hot-start ultra-fidelity 2× Master Mix kit (New England Biolabs, M0494S). The final concentration of each primer was 0.5 M, 10 μl of Q5 2× Master Mix was added, and then water was added to a total volume of 50 μl, and then PCR was performed according to the manufacturer's guidelines. On the basis of the above reaction system, for the DNA sequences of SEQ ID NOs. 7-11, an additional 1 μl of cell genome extract was added, and the final volume was made up to 50 μl.

### Example 3

### Construction and Purification of the dCas9 Protein Coupled with FLAG

1 Protein expression

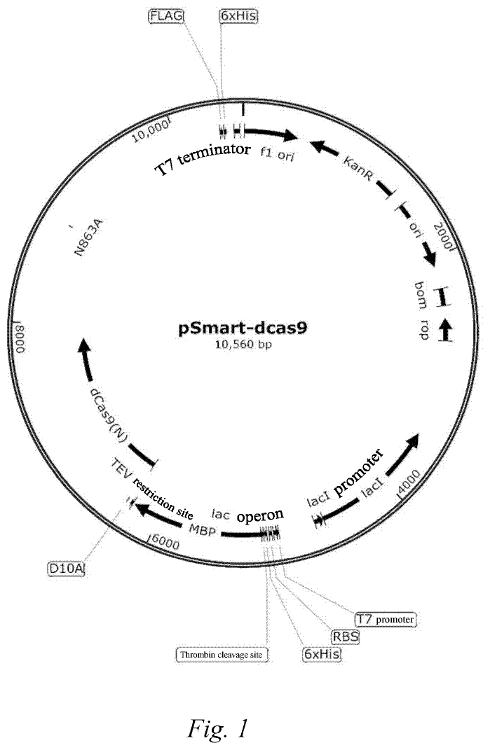

1.1 Construction of expression vector: pSMART-his-S.P.dCas9-FLAG. The following was a schematic diagram of the vector loop, and the his-S P dCas9-FLAG gene fragment (SEQ ID No: 34) was integrated into the pSMART vector by EcoRI and BamHI restriction endonucleases (see FIG. 1); if other linker sequences were used, then FLAG (FLAG sequence: GATTACAAGGATGACGATGACAAG) in the vector was replaced with the corresponding sequence, such as AVI-Tag, and other steps for expressing and purifying a protein were the same:

1.2 Selection of expression strains: Rosetta2 (NOVAGEN, 17400)

1.3 Culture conditions: Shaking culture

Instruments and consumables: shaker, LB medium (Biotechnology Co., Ltd, A507002)

Steps:

a plate (Rosetta/BL21) was taken out from the refrigerator at 4° C. to be activate overnight at 37° C.; transfer: 500 ml of LB was inoculated with 4 ml of bacterial solution and incubated at 37° C. for 4 hours, OD600=0.8, and taken out at room temperature. The shaker temperature was cooled to 18° C., 500 ul of IPTG (isopropyl-β-D-thiogalactopyranoside, Tiangen RT108-01) was added, and induced overnight with an induction time of 16 hours.

2 Protein purification

2.1 Cell harvest and lysis

Instruments and consumables: centrifuge, homogenizer, high-speed centrifuge, 0.45 μm filter membrane (Merck Millipore, SLHV033) and lysate

Steps: The cells were collected by centrifugation at 5000 rpm for 30 minutes, added with lysate to resuspend, and crushed by a homogenizer. The supernatant and precipitate were separated by high speed centrifugation. The supernatant was passed through a 0.45 μm filter membrane and was ready for the next step of chromatographic purification.

2.2 Chromatography column purification process:

Instruments: AKTA-purify, chromatography column, chromatography solution

2.2.1 Histone affinity chromatography

Steps: A 20 ml histone affinity chromatography column was directly packed with a purchased medium (GE Life Sciences, 17-5318-02), and equilibrated with a 0.5% solution B (20 mM Tris-Hcl, PH=8.0; 250 mM NaCl; 1 M imidazole). A purified protein solution was obtained according to the AKTA instrument instructions.

2.2.2 Cation exchange chromatography

Steps: A 20 ml cation exchange chromatography column (GE Life Sciences, 17-0407-01) was used, and equilibrated with a 0.5% solution B (20 mM HEPES-KOH PH=7.5; 1 M KCL). A purified protein solution was obtained according to the AKTA instrument instructions.

3 Concentration and liquid change

Instruments and consumables: 30 kD concentration tube (Merck Millipore, UFC903096), low temperature high speed centrifuge

Steps: The protein solution was added to a concentration tube, and centrifuged at 4° C., 5000 rpm for 40 minutes; the concentration tube was taken out, the penetrating solution was removed, and 15 ml of protein preservation solution (20 mM Hepes, PH=7.5; 150 mM KCl; 1% sucrose; 30% glycerol; 1 mM dithiothreitol (DTT)) was added, and the mixture was centrifuged at 4° C. for 40 minutes; and the above steps were repeated 3 times to obtain the final his-S.P.dCas9-FLAG protein solution, which would be stored at −80° C. after sub-packing.

### Example 4

### Binding of Cell-Free DNA to the Cas Protein

Under normal circumstances, the CRISPR/Cas system recognizes and cleaves specific DNA sequences based on the guidance of the PAM region (sequence: NGG, N may be A/T/C/G) and RNA. In order to verify whether the direct binding of the Cas protein and a variant thereof to a nucleic acid also depended on the PAM region, we prepared DNA sequences of SEQ ID NOs. 1-6. SEQ ID NOs.1-3 were 100 bp, SEQ ID NOs. 4-6 were 200 bp, of which there was no PAM region (NGG) corresponding to the Cas9 in SEQ ID NO. 1 and SEQ ID NO. 4, there was one PAM region on SEQ ID NO. 2 and SEQ ID NO. 5 respectively, and there was 3 PAM regions on SEQ ID NO. 3 and SEQ ID NO. 6 respectively. The dCas9 protein used in the reaction was the same as that in Examples 5, 6 and 7, which was the purified FLAG-dCas9 protein produced in Example 3. The reaction conditions were as followed:

reaction buffer (BA): 50 mM tris-hydroxymethylaminomethane hydrochloride (Tris-HCl, pH 7.4); 150 mM sodium chloride (NaCl)

reaction system:

total volume: 20 μl

dCas9 protein: 2 μg

DNA: 0.25 μg

Nuclease-free double distilled water: the volume was made up to 20 μl

Reaction process and result:

the above reaction system was added into a 200 μl PCR tube and mixed well, reacted at room temperature or 37° C. for 15 minutes to 1 hour. After the reaction was completed, a non-denaturing gel was run in a BioRad electrophoresis tank at 80V and 400 mA for 80 minutes. After removing the non-denaturing gel, the nucleic acid was stained with DuRed (Shanghai Yeasen Bio Technologies Co., Ltd., 10202ES76), and then a picture (FIG. 2) was taken by using a gel imager. FIG. 2 showed the binding electrophoresis band of cell-free DNA (SEQ ID NO. 7) of about 500 bp in length to a dCas protein. Lane 1 was a band of cell-free DNA itself and was a negative control. Lane 2 showed the situation after the dCas9 and cell-free DNA (SEQ ID NO. 7) were bound. In the negative control, the band of cell-free DNA was clear and complete, and the electrophoresis was successful. However, due to the binding of cell-free DNA to dCas9 in the second lane, DNA could not be electrophoresed normally, and almost all DNA was left in the lane well, therefore there was no obvious band at the corresponding position. In order to calculate the binding proportion (E1) of dCas9 to DNA, we used the following formula:

$$E 1 = \frac{\begin{matrix}Band brightness of lane 1 - \\ Band brightness at the corresponding position of lane 2\end{matrix}}{Band brightness of lane 1}$$

The formula used the band brightness of lane 1 as the benchmark for the total DNA amount, and the band brightness at the corresponding position of lane 2 as the amount of DNA not bound to the dCas9. So after the molecular calculation, it was the amount of DNA bound to the dCas9 protein, which was then divided by the total DNA amount to obtain the binding efficiency of the dCas9 to DNA, and the calculated binding efficiency of the dCas9 protein to DNA was 97.4%. Using the same experiment and calculation method, Table 3 summarized the dCas9-DNA binding proportions corresponding to different cell-free DNA lengths. The results showed that the dCas9 could efficiently bind to the above 11 types of DNA without difference, showing good potential for isolating nucleic acid from a solution.

TABLE 3
dCas9-DNA binding proportion corresponding to each DNA

| | SEQ ID NO. | | | | | | | | | | |
|---|---|---|---|---|---|---|---|---|---|---|---|
| | 1 | 2 | 3 | 4 | 5 | 6 | 7 | 8 | 9 | 10 | 11 |
| DCAS9-DNA Binding | 97.1% | 96.9% | 96.1% | 96.9% | 97.0% | 96.8% | 97.1% | 97.4% | 98.4% | 97.2% | 96.4% |
| proportion (E1) | | | | | | | | | | | |

### Example 5

### After the dCas9 Protein Coupled with FLAG was Bound to Cell-Free DNA in a solution, it was Bound to Solid Beads and Isolated

reaction system:

total volume: 100 μl

dCas9 protein: 10 μg

DNA (SEQ ID NO. 7, 8, 9, 11, representing the length of different cell-free DNA): 0.25 μg of each DNA

Nuclease-free double distilled water: the volume was made up to 100 μl

the above reaction system was added into a 1.5 ml PCR tube and mixed well, reacted at room temperature or 37° C. for 15 minutes to 1 hour.

15 μl of anti-FLAG affinity gel beads (Bimake, B23101) was added into a 1.5 ml centrifuge tube, 150 μl of the buffer solution (BA) in Example 4 was added, and the mixture was mixed well and centrifuged at 5000 RPM for 30 seconds. The supernatant was discarded, and the above step was repeated once. After the supernatant was discarded, the above 100 μl reaction system was added to the washed affinity gel beads, inverted and mixed well at room temperature, and reacted for 1 hour.

After the reaction was completed, the mixture was centrifuged at 5000 RPM for 30 seconds, the supernatant was discarded. 150 μl of the buffer solution (BA) in Example 4 was added, mixed well and centrifuged at 5000 RPM for 30 seconds. The supernatant was discarded, and the above step was repeated once. After the supernatant was discarded, 30 μl of proteinase K (Tiangen Biotech Co. Ltd, RT403) was added to the gel beads, and 20 μl of BA was added. After the mixture was mixed well, it was heated in a 55° C. water bath for 30 minutes, and centrifuged at 5000 RPM for 30 seconds, and the supernatant was taken to obtain the isolated cell-free DNA. After gel electrophoresis and comparison with the positive control (0.25 μg of DNA was dissolved in 30 μl of proteinase K and 20 μl of BA solution), the DNA isolation efficiency E2 was calculated according to the following formula and summarized in Table 4:

$$E 2 = \frac{Brightness of DNA capture lane}{Brightness of positive control lane}$$

TABLE 4
dCas9-DNA isolation efficiency corresponding to each DNA

| | SEQ ID NO:. | | | |
|---|---|---|---|---|
| | 7 | 8 | 9 | 11 |
| DNA isolation efficiency (E2) | 79.5% | 77.2% | 77.6% | 70.9% |

### Example 6

### After the dCas9 Protein Coupled with FLAG was Bound to the Solid Beads, Cell-Free DNA was Bound and Isolated from the Solution

The first step: 15 μl of anti-FLAG affinity gel beads (GenScript, L00439-1) was added into a 1.5 ml centrifuge tube, 150 μl of the buffer solution (BA) in Example 4 was added, and the mixture was mixed well and centrifuged at 5000 RPM for 30 seconds. The supernatant was discarded, and the above step was repeated once. After the supernatant was discarded, 500 μl of BA was added to the gel beads and mixed well. Then 1 μl of 10 μg/μl dCas9 protein solution was added to the mixture, inverted and mixed well at room temperature, and reacted for 1 hour.

The second step: After the reaction was completed, the mixture was centrifuged at 5000 RPM for 30 seconds, the supernatant was discarded. 150 μl of the buffer solution (BA) in example 4 was added, mixed well and centrifuged at 5000 RPM for 30 seconds. The supernatant was discarded, and the above step was repeated once. The washed gel beads were added to 4 tubes of 1 ml solution containing cell-free DNA with different lengths (SEQ ID NOs. 7, 8, 9 and 11 were used for cell-free DNA, representing the lengths of different cell-free DNA were 957, 486, 166 and 2655 bp, respectively) respectively, the mixture was inverted, mixed well and reacted at room temperature or 37° C. for 15 minutes to 1 hour.

The third step: After the reaction was completed, the mixture was centrifuged at 5000 RPM for 30 seconds, the supernatant was discarded. 150 μl of the buffer solution (BA) in Example 4 was added, mixed well and centrifuged at 5000 RPM for 30 seconds. The supernatant was discarded, and the above step was repeated once. After the supernatant was discarded, 30 μl of proteinase K (Tiangen Biotech Co. Ltd, RT403) was added to the gel beads, and 20 μl of BA was added. After the mixture was mixed well, it was heated in a 55° C. water bath for 30 minutes, and centrifuged at 5000 RPM for 30 seconds, and the supernatant was taken to obtain the isolated cell-free DNA.

In order to compare with QIAGEN's existing kits, cell-free DNA in a solution was extracted from the 1 ml of cell-free DNA solution in the second step by using the QIAamp® Circulating Nucleic Acid kit (QIAGEN Inc.) according to the steps in the instruction manual.

Positive control: The same amount of DNA (SEQ ID NOs. 7, 8, 9 and 11 were used for cell-free DNA, representing the lengths of different cell-free DNA were 957, 486, 166 and 2655 bp, respectively) was dissolved in 30 μl of proteinase K (Tiangen Biotech Co. Ltd, RT403), and 20 μl of BA was added.

The Agilent 2100 High Sensitivity DNA Analysis Kit (Cat. 5067-4626) was used to perform DNA quantitative analysis on DNA captured by the present invention, DNA captured by the Qiagen kit and the positive control. The results of amount and percentage of captured DNA were summarized in Table 4. The extraction efficiency was the ratio of the amount of DNA captured by the present invention or Qiagen kit in Table 4 to the amount of the positive control DNA (i.e., the percentage of captured DNA). The cell-free DNA extraction efficiency of the present invention was close to 80%, and the extraction efficiency of the QIAamp® Circulating Nucleic Acid kit was less than 50%, therefore the present invention increased the cell-free DNA extraction efficiency by about 60%.

TABLE 4
Comparison of the extraction efficiencies of cell-free DNA with various lengths
by using the present invention and QIAamp ® Circulating Nucleic Acid kit

| | The amount | The amount | DNA | The | The |
|---|---|---|---|---|---|
| | of DNA | of DNA | amount | extraction | extraction |
| DNA | captured by the | captured by the | of the positive | efficiency of | efficiency |
| size | present invention | Qiagen kit | control | the present | of the |
| [BP] | [pg/μl] | [pg/μl] | [pg/μl] | invention | Qiagen kit |
| 166 | 1,025.24 | 631.79 | 1,305.28 | 78.55% | 48.40% |
| 486 | 1,012.37 | 549.34 | 1,261.83 | 80.23% | 38.22% |
| 957 | 818.39 | 308.745 | 1077.58 | 75.95% | 28.65% |
| 2655 | 711.04 | 436.435 | 1179.08 | 60.30% | 28.65% |

### Example 7

### After the Cpf1 (Cas12a) Protein with His-Tag was Bound to the Solid Beads, Cell-Free DNA was Bound and Isolated from the Solution

The first step: The Cpf1 (Cas12a) protein with His-Tag (IDT, Alt-R® As Cas12a (Cpf1) V3, Cat. No. 1081068) was linked to the His-Tag isolation magnetic beads (Dynabeads™ His-Tag Isolation and Pulldown, Cat. No. 10103D). 300 μl of Dynabeads were added into a 1.5 ml centrifuge tube, and 1 ml of pH=7.4 PBS was added and mixed well. The centrifuge tube was placed in the corresponding sample hole of a magnetic separator (Beaver Company, Cat. No. 60201), and left to stand for two minutes, so that the magnetic beads were adsorbed and gathered on the wall of the tube, and the solution was restored to clarification. The supernatant was carefully aspirated, and the above steps were repeated twice. 40 μl of the Cpf1 protein was mixed well with the Dynabeads, 1 ml of pH=7.4 PBS was added, and the mixture was put on a mixer at room temperature to rotate for 20 minutes to 2 hours. The centrifuge tube was placed in the corresponding sample hole of a magnetic separator, and left to stand for two minutes. The supernatant was discarded, 500 μl of the buffer solution (BA) in Example 4 was added and mixed well. The centrifuge tube was placed in the corresponding sample hole of a magnetic separator, and left to stand for two minutes. The supernatant was discarded, and the above step was repeated once. 10 to 100 μl of the washed Dynabeads were added to 1 ml of a solution containing DNA of SEQ ID NO. 9, the mixture was inverted, mixed well and reacted at room temperature or 37° C. for 15 minutes to 6 hours.

The second step: After the reaction was completed, the centrifuge tube containing the reaction solution and Dynabeads was placed in the corresponding sample hole of a magnetic separator, and left to stand for two minutes. The supernatant was discarded, and 150 μl of the buffer solution (BA) in example 4 was added. The centrifuge tube was placed in the corresponding sample hole of a magnetic separator, and left to stand for two minutes. The supernatant was discarded, and the above step was repeated once. After the supernatant was discarded, 30 μl of proteinase K (Tiangen Biotech Co. Ltd, RT403) was added to the Dynabeads, and 20 μl of BA was added. After the mixture was mixed well, it was heated in a 55° C. water bath for 30 minutes. The centrifuge tube was placed in the corresponding sample hole of a magnetic separator, and left to stand for two minutes, and the centrifuge tube was placed in the sample hole corresponding to the magnetic separator, left to stand for two minutes, and the supernatant was taken to obtain the isolated cell-free DNA.

In order to compare with QIAGEN's existing kits, cell-free DNA in plasma was extracted from the 1 ml of cell-free DNA solution in the third step by using the QIAamp® Circulating Nucleic Acid kit (QIAGEN Inc.) according to the steps in the instruction manual.

Positive control: The same amount of DNA (SEQ ID NO. 9 was used for cell-free DNA, representing the length of cell-free DNA was 166 bp) was dissolved in 30 μl of proteinase K (Tiangen Biotech Co. Ltd, RT403), and 20 μl of BA was added.

The Agilent 2100 High Sensitivity DNA Analysis Kit (Cat. 5067-4626) was used to perform DNA quantitative analysis on the DNA captured by the present invention, the DNA captured by the Qiagen kit and the positive control. The results of amount and percentage of the captured DNA were summarized in Table 5. The extraction efficiency was the ratio of the amount of the DNA captured by the present invention or Qiagen kit in Table 5 to the amount of the positive control DNA (i.e., the percentage of the captured DNA). The cell-free DNA extraction efficiency of the present invention was close to 80%, and the extraction efficiency of the QIAamp® Circulating Nucleic Acid kit was only 51 %, therefore the present invention increased the cell-free DNA extraction efficiency from plasma by about 54.7%.

TABLE 5
Comparison of the extraction efficiencies of cell-free DNA with 160 bp
by using the present invention and QIAamp ® Circulating Nucleic Acid kit

| | The amount | The amount | DNA | The | The |
|---|---|---|---|---|---|
| | of DNA | of DNA | amount of | extraction | extraction |
| DNA | captured by the | captured by the | the positive | efficiency of | efficiency |
| size | present invention | QIAGEN KIT | control | the present | of the |
| [BP] | [pg/μl] | [pg/μl] | [pg/μl] | invention | QIAGEN KIT |
| 166 | 1,018.62 | 658.34 | 1,293.72 | 78.74% | 50.89% |

### Example 8

### After the dCas9 protein coupled with AVI-Tag was bound to the solid beads, exogenous cell-free DNA was bound and isolated from plasma

The first step: For purification of the his-S.P.dCas9-AVI protein, example 3 was referred, and the FLAG sequence was replaced with the AVI-Tag sequence (DNA sequence: ggcctgaacgatatttttgaagcgcagaaaattgaatggcatgaa).

The second step: The AVI-Tag-dCas9 protein was biotinylated by using the BirA-500 kit (Cat. BirA500) from AViDITY according to the instructions of the kit. Corresponding reactants were added to a 1.5 ml centrifuge tube to react, and the reaction system was as follows:

10×BiomixA 8 μl

10×BiomixB 8 μl

AVI-Tag-dCas9 protein 50 μl

BirA enzyme 0.8 μl

Ultra-pure water The total volume was made up to 80 μl

And then the reaction solution was reacted at room temperature for 1 hour or overnight.

The third step: The biotinylated protein was linked to Streptavidin gel beads. 400 μl of the GenScript Streptavidin gel beads (Cat. No. L00353) was placed in a 1.5 ml centrifuge tube, 1 ml of pH=7.4 PBS was added, and the mixture was centrifuged at 5000 g for 30 seconds at room temperature. After the centrifuge tube was taken out, the supernatant was carefully aspirated, and the above step was repeated twice. 80 μl of the biotinylated protein and the Streptavidin gel beads were mixed well, 1 ml of pH=7.4 PBS was added, and the mixture was put on a mixer at room temperature to rotate for 20 minutes to 2 hours, and centrifuged at 5000 g at room temperature for 30 seconds. The supernatant was discarded. 500 μl of the buffer solution (BA) in example 4 was added, mixed well and centrifuged at 5000 RPM for 30 seconds. The supernatant was discarded, and the above step was repeated once. 10 to 100 μl of the washed gel beads were added to 1 ml of plasma containing DNA of SEQ ID NO. 9, the mixture was inverted, mixed well and reacted at room temperature or 37° C. for 15 minutes to 6 hours.

The fourth step: After the reaction was completed, the mixture was centrifuged at 5000 RPM for 30 seconds, the supernatant was discarded. 150 μl of the buffer solution (BA) in example 4 was added, mixed well and centrifuged at 5000 RPM for 30 seconds. The supernatant was discarded, and the above step was repeated once. After the supernatant was discarded, 30 μl of proteinase K (Tiangen Biotech Co. Ltd, RT403) was added to the gel beads, and 20 μl of BA was added. After the mixture was mixed well, it was heated in a 55° C. water bath for 30 minutes, and centrifuged at 5000 RPM for 30 seconds, and the supernatant was taken to obtain the isolated cell-free DNA.

In order to compare with QIAGEN's existing kits, cell-free DNA in plasma was extracted from the 1 ml of cell-free DNA solution in the third step by using the QIAamp® Circulating Nucleic Acid kit (QIAGEN Inc.) according to the steps in the instruction manual.

Positive control: The same amount of DNA (SEQ ID NO. 9 was used for cell-free DNA, representing the length of cell-free DNA was 166 bp) was dissolved in 30 μl of proteinase K (Tiangen Biotech Co. Ltd, RT403), and 20 μl of BA was added.

The Agilent 2100 High Sensitivity DNA Analysis Kit (Cat. 5067-4626) was used to perform DNA quantitative analysis on the DNA captured by the present invention, the DNA captured by the Qiagen kit and the positive control. The results of amount and percentage of the captured DNA were summarized in Table 6. The extraction efficiency was the ratio of the amount of the DNA captured by the present invention or Qiagen kit in Table 6 to the amount of the positive control DNA (i.e., the percentage of the captured DNA). The cell-free DNA extraction efficiency of the present invention was close to 78%, and the extraction efficiency of the QIAamp® Circulating Nucleic Acid kit was only 51%, therefore the present invention increased the cell-free DNA extraction efficiency from plasma by about 53%.

TABLE 6
Comparison of the extraction efficiencies of cell-free DNA with 160 bp
by using the present invention and QIAamp ® Circulating Nucleic Acid kit

| | The amount | The amount | DNA | The | The |
|---|---|---|---|---|---|
| | of DNA | of DNA | amount of | extraction | extraction |
| DNA | captured by the | captured by the | the positive | efficiency of | efficiency |
| size | present invention | QIAGEN KIT | control | the present | of the |
| [BP] | [pg/μl] | [pg/μl] | [pg/μl] | invention | QIAGEN KIT |
| 166 | 1,007.05 | 674.35 | 1,305.45 | 77.14% | 51.66% |

### Example 9

### After the Cas9 protein with His-Tag was bound to the solid beads, exogenous cell-free DNA was bound and isolated from plasma

The first step: The Cas9 protein with His-Tag (IDT, Alt-R® Sp Cas9 Nuclease V3, Cat. No. 1081058) was linked to the His-Tag isolation magnetic beads (Dynabeads™ His-Tag Isolation and Pulldown, Cat. No. 10103D). 300 μl of Dynabeads were added into a 1.5 ml centrifuge tube, and 1 ml of pH=7.4 PBS was added and mixed well. The centrifuge tube was placed in the corresponding sample hole of a magnetic separator (Beaver Company, Cat. No. 60201), and left to stand for two minutes, so that the magnetic beads were adsorbed and gathered on the wall of the tube, and the solution was restored to clarification. The supernatant was carefully aspirated, and the above steps were repeated twice. 40 μl of the Cas9 protein was mixed well with the Dynabeads, 1 ml of pH=7.4 PBS was added, and the mixture was put on a mixer at room temperature to rotate for 20 minutes to 2 hours. The centrifuge tube was placed in the corresponding sample hole of a magnetic separator, and left to stand for two minutes. The supernatant was discarded, 500 μl of the buffer solution (BA) in example 4 was added and mixed well. The centrifuge tube was placed in the corresponding sample hole of a magnetic separator, and left to stand for two minutes. The supernatant was discarded, and the above step was repeated once. 10 to 100 μl of the washed Dynabeads were added to 1 ml of plasma containing DNA of SEQ ID NO. 9, the mixture was inverted, mixed well and reacted at room temperature or 37° C. for 15 minutes to 6 hours.

The second step: After the reaction was completed, the centrifuge tube containing plasma and Dynabeads was placed in the corresponding sample hole of a magnetic separator, and left to stand for two minutes. The supernatant was discarded, and 150 μl of the buffer solution (BA) in example 4 was added. The centrifuge tube was placed in the corresponding sample hole of a magnetic separator, and left to stand for two minutes. The supernatant was discarded, and the above step was repeated once. After the supernatant was discarded, 30 μl of proteinase K (Tiangen Biotech Co. Ltd, RT403) was added to the Dynabeads, and 20 μl of BA was added. After the mixture was mixed well, it was heated in a 55° C. water bath for 30 minutes. The centrifuge tube was placed in the corresponding sample hole of a magnetic separator, and left to stand for two minutes, and the centrifuge tube was placed in the sample hole corresponding to the magnetic separator, left to stand for two minutes, and the supernatant was taken to obtain the isolated cell-free DNA.

In order to compare with QIAGEN's existing kits, cell-free DNA in plasma was extracted from the 1 ml of cell-free DNA solution in the third step by using the QIAamp® Circulating Nucleic Acid kit (QIAGEN Inc.) according to the steps in the instruction manual.

Positive control: The same amount of DNA (SEQ ID NO. 9 was used for cell-free DNA, representing the length of cell-free DNA was 166 bp) was dissolved in 30 μl of proteinase K (Tiangen Biotech Co. Ltd, RT403), and 20 μl of BA was added.

The Agilent 2100 High Sensitivity DNA Analysis Kit (Cat. 5067-4626) was used to perform DNA quantitative analysis on the DNA captured by the present invention, the DNA captured by the Qiagen kit and the positive control. The results of amount and percentage of the captured DNA were summarized in Table 7. The extraction efficiency was the ratio of the amount of the DNA captured by the present invention or Qiagen kit in Table 7 to the amount of the positive control DNA (i.e., the percentage of the captured DNA). The cell-free DNA extraction efficiency of the present invention exceeded 85%, and the extraction efficiency of the QIAamp® Circulating Nucleic Acid kit was only 55%, therefore the present invention increased the cell-free DNA extraction efficiency from plasma by about 54.5%.

TABLE 7
Comparison of the extraction efficiencies of cell-free DNA with 160 bp
by using the present invention and QIAamp ® Circulating Nucleic Acid kit

| | The amount | The amount | DNA | The | The |
|---|---|---|---|---|---|
| | of DNA | of DNA | amount of | extraction | extraction |
| DNA | captured by the | captured by the | the positive | efficiency of | efficiency |
| size | present invention | QIAGEN KIT | control | the present | of the |
| [BP] | [pg/μl] | [pg/μl] | [pg/μl] | invention | QIAGEN KIT |
| 170 | 1,160.66 | 754.54 | 1,363.28 | 85.14% | 55.35% |

### Example 10

### After the dCas9 Protein Coupled with AVI-Tag was Bound to the Solid Beads, Endogenous Cell-Free DNA was Bound and Isolated from Plasma

The first step and the second step were the same as the first step and the second step in example 8, and a biotinylated dCas9 protein was obtained.

The third step: The biotinylated protein was linked to Streptavidin gel beads. 400 μl of the GenScript Streptavidin gel beads (Cat. No. L00353) was placed in a 1.5 ml centrifuge tube, 1 ml of pH=7.4 PBS was added, and the mixture was centrifuged at 5000 g for 30 seconds at room temperature. After the centrifuge tube was taken out, the supernatant was carefully aspirated, and the above step was repeated twice. 80 μl of the biotinylated protein and the Streptavidin gel beads were mixed well, 1 ml of pH=7.4 PBS was added, and the mixture was put on a mixer at room temperature to rotate for 20 minutes to 2 hours, and centrifuged at 5000 g at room temperature for 30 seconds. The supernatant was discarded. 500 μl of the buffer solution (BA) in example 4 was added, mixed well and centrifuged at 5000 RPM for 30 seconds. The supernatant was discarded, and the above step was repeated once. 10 to 100 μl of the washed gel beads were added to 1 ml of plasma, the mixture was inverted, mixed well and reacted at room temperature or 37° C. for 15 minutes to 6 hours.

The fourth step: After the reaction was completed, the mixture was centrifuged at 5000 RPM for 30 seconds, the supernatant was discarded. 150 μl of the buffer solution (BA) in example 4 was added, mixed well and centrifuged at 5000 RPM for 30 seconds. The supernatant was discarded, and the above step was repeated once. After the supernatant was discarded, 30 μl of proteinase K (Tiangen Biotech Co. Ltd, RT403) was added to the gel beads, and 20 μl of BA was added. After the mixture was mixed well, it was heated in a 55° C. water bath for 30 minutes, and centrifuged at 5000 RPM for 30 seconds, and the supernatant was taken to obtain the isolated cell-free DNA.

In order to compare with QIAGEN's existing kits, cell-free DNA in plasma was extracted from the 1 ml of cell-free DNA solution in the third step by using the QIAamp® Circulating Nucleic Acid kit (QIAGEN Inc.) according to the steps in the instruction manual.

The Agilent 2100 High Sensitivity DNA Analysis Kit (Cat. 5067-4626) was used to perform DNA quantitative analysis on DNA captured by the present invention and DNA captured by the Qiagen kit. The results of captured DNA amount were summarized in Table 8. The cell-free DNA extraction amount of the present invention was close to 28.49 pg/μl, and the extraction efficiency of the QIAampg Circulating Nucleic Acid kit was only 24.68 pg/μ1, therefore the present invention increased the endogenous cell-free DNA extraction efficiency from plasma by about 15.4%.

TABLE 8
Comparison of the extraction amounts of cell-free DNA
in plasma by using the present invention and QIAamp ® Circulating
Nucleic Acid kit

| | The amount | The amount | The extraction |
|---|---|---|---|
| | of DNA | of DNA | efficiency |
| DNA | captured by the | captured by the | of the |
| size | present invention | QIAGEN KIT | present invention |
| [BP] | [pg/μl] | [pg/μl] | increased by |
| 177 | 28.49 | 24.68 | 15.4% |

1. A method for isolating DNA from a DNA solution by using a Cas protein, comprising:
mixing a DNA solution with a Cas protein system to form a complex resulting from binding of the Cas protein system with DNA in the DNA solution; and
isolating DNA from the complex formed by the binding of the Cas protein system with DNA.

2. (canceled)

3. The method of claim 1, further comprising enriching the complex formed by mixing the DNA solution with the Cas protein system prior to isolating the DNA from the complex.

4. The method of claim 1, wherein the Cas protein system is a Cas protein coupled with a plate-like, film-like or columnar solid support.

5. The method of claim 3, wherein the Cas protein system is a Cas protein coupled with a bead-shaped solid support.

6. The method of claim 3, wherein the Cas protein system is a Cas fusion protein formed by the Cas protein and at least one linker sequence.

7. The method of claim 6, wherein in the step of enriching the complex, a solid support coupled with an affinity molecule is added to the DNA solution, then the enrichment is performed by centrifugation or magnetism.

8. The method of claim 6, wherein a solid support coupled with an affinity molecule is added in the step of mixing the DNA solution, then the step of enriching the complex is performed, after incubation.

9. The method of claim 1, wherein the DNA is double-stranded DNA.

10. The method of claim 4, wherein the Cas protein system is formed by coupling the Cas protein to a solid support through a chemical covalent bond.

11. The method of claim 6, wherein the Cas protein system is formed by combining the Cas fusion protein with the affinity molecule on the solid support.

12. The method of claim 4, wherein the solid support is a solid substance capable of forming a chemical bond with an amino acid residue.

13. The method of claim 10, wherein the solid support is selected from at least one of solid substances made of gel materials, magnetic materials, cellulose materials, silica gel materials, glass materials, and artificial high-molecular polymers.

14. The method of claim 5, wherein the bead-shaped solid support is a magnetic bead, gel bead, and/or silica gel bead.

15. The method of claim 6, wherein the linker sequence and the affinity molecule are selected from one of the following combinations of ligand and receptor: enzyme and substrate, antigen and antibody, and biotin and avidin.

16. The method of claim 15, wherein the enzyme and substrate are selected from glutathione transferase and glutathione.

17. The method of claim 15, wherein the antigen and antibody are selected from the group of: a protein A or a fragment thereof which retains the Fc region-binding function and an immunoglobulin or Fc or Fab fragments thereof, a protein G or a fragment thereof which retains the Fc region-binding function and an immunoglobulin or Fc or Fab fragments thereof, a histidine tag and an anti-histidine tag, a polyhistidine tag and an anti-polyhistidine tag, and a FLAG tag and an anti-FLAG tag.

18. The method of claim 15, wherein the biotin and avidin are selected from the group of: biotin and avidin or streptavidin, biotin-linked biotin receptor protein and avidin or streptavidin, and Strep-tag and avidin or streptavidin.

19-25. (canceled)

26. The method of claim 3, wherein the Cas protein is a natural Cas protein, a mutant Cas protein which loses the gRNA binding ability, or a mutant Cas protein which loses the nuclease activity.

27-29. (canceled)

30. A kit for isolating DNA from a solution, comprising components a) a Cas protein system, and b) a DNA eluent.

31. The kit of claim 30, wherein component a) the Cas protein system is a solution system containing a Cas protein coupled with a solid support, or is a solution system containing a Cas fusion protein and a solid support coupled with an affinity molecule.

32-33. (canceled)


SEQUENCE LISTING (34 sequences)

SEQ ID NO: 1 - DNA, 100 bp, Artificial Sequence
  Random DNA Sequence
ctgcgtaaacgtcgctgtgctagctcttttgcagtcacagcttttcgtgcatgagtacgtattttgaaactcaagatcgcattcatgcgtcttcacgtga

SEQ ID NO: 2 - DNA, 100 bp, Artificial Sequence
  Random DNA Sequence
ctgcgtaaacgtcgctgtgctagctcttttgcaggcacagcttttcgtgcatgagtacgtattttgaaactcaagatcgcattcatgcgtcttcacgtga

SEQ ID NO: 3 - DNA, 100 bp, Artificial Sequence
  Random DNA Sequence
ctgcgtaaacgtcgcggtgctagctcttttgcaggcacagcttttcgtgcatgagtatggattttgaaactcaagatcgcattcatgcgtcttcacgtga

SEQ ID NO: 4 - DNA, 200 bp, Artificial Sequence
  Random DNA Sequence
ctgcttcgtgcatgagtacgtattctctttacgtgactgcgtaagtaaacgtcgctgtgctagtgcagtcacagttgaaactcaagatcgcattcaagctctttactgcttacgtgactgcgttgcagtcacagcttagtacgtattgcgtcttcagtgcatgttgaaactcaagatcgcattcatgcgtcttcacgtga

SEQ ID NO: 5 - DNA, 200 bp, Artificial Sequence
  Random DNA Sequence
ctgcttcgtgcatgagtacgtattctctttacgtgactgcgtaagtaaacgtcgctgtgctagtgcagtcacagttgaaactcaagatcgcattcaaggtctttactgcttacgtgactgcgttgcagtcacagcttagtacgtattgcgtcttcagtgcatgttgaaactcaagatcgcattcatgcgtcttcacgtga

SEQ ID NO: 6 - DNA, 200 bp, Artificial Sequence
  Random DNA Sequence
ctgcttcgtgcatgcggacgtattctctttacgtgactgcgtaagtaaacgtcgctgtgctagtgcagtcacagttgaaactcaagatcgcattcaaggtcgttactgcttacgtgactgcgttgcagtcacagcttagtacgtattgcgtcttcagtgcatgttgaaactcaaggtcgcattcatgcgtcttcacgtga

SEQ ID NO: 7 - DNA, 957 bp, Homo sapiens
gccctcatgatattttaaaacacagcatcctcaaccttgaggcggaggtcttcataacaaagatactatcagttcccaaactcagagatcaggtgactccgactcctcctttatccaatgtgctcctcatggccactgttgcctgggcctctctgtcatggggaatccccagatgcacccaggaggggccctctcccactgcatctgtcacttcacagccctgcgtaaacgtccctgtgctaggtcttttgcaggcacagcttttcctccatgagtacgtattttgaaactcaagatcgcattcatgcgtcttcacctggaaggggtccatgtgcccctccttctggccaccatgcgaagccacactgacgtgcctctccctccctccaggaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaaggtaatcagggaagggagatacggggaggggagataaggagccaggatcctcacatgcggtctgcgctcctgggatagcaagagtttgccatggggatatgtgtgtgcgtgcatgcagcacacacacattcctttattttggattcaatcaagttgatcttcttgtgcacaaatcagtgcctgtcccatctgcatgtggaaactctcatcaatcagctacctttgaagaattttctctttattgagtgctcagtgtggtctgatgtctctgttcttatttctctggaattctttgtgaatactgtggtgatttgtagtggagaaggaatattgcttcccccattcaggacttgataacaaggtaagcaagccaggccaaggc

SEQ ID NO: 8 - DNA, 486 bp, Homo sapiens
ctctcccactgcatctgtcacttcacagccctgcgtaaacgtccctgtgctaggtcttttgcaggcacagcttttcctccatgagtacgtattttgaaactcaagatcgcattcatgcgtcttcacctggaaggggtccatgtgcccctccttctggccaccatgcgaagccacactgacgtgcctctccctccctccaggaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaaggtaatcagggaagggagatacggggaggggagataaggagccaggatcctcacatgcggtctgcgctcctgggatagcaagagtttgccatggggatatg

SEQ ID NO: 9 - DNA, 166 bp, Homo sapiens
ctccaggaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgc

SEQ ID NO: 10 - DNA, 609 bp, Homo sapiens
tctccacaaggaggcatggaaaggctgtagttgttcacctgcccaagaactaggaggtctggggtgggagagtcagcctgctctggatgctgaaagaatgtctgtttttccttttagaaagttcctgtgatgtcaagctggtcgagaaaagctttgaaacaggtaagacaggggtctagcctgggtttgcacaggattgcggaagtgatgaacccgcaataaccctgcctggatgagggagtgggaagaaattagtagatgtgggaatgaatgatgaggaatggaaacagcggttcaagacctgcccagagctgggtggggtctctcctgaatccctctcaccatctctgactttccattctaagcactttgaggatgagtttctagcttcaatagaccaaggactctctcctaggcctctgtattcctttcaacagctccactgtcaagagagccagagagagcttctgggtggcccagctgtgaaatttctgagtcccttagggatagccctaaacgaaccagatcatcctgaggacagccaagaggttttgccttctttcaagacaagcaacagtactcacataggctgtgggcaatggtcctgtc

SEQ ID NO: 11 - DNA, 2655 bp, Homo sapiens
cgttccagaagcggcagaagcttacctccgagggtgccgccaagctcctgctagacaccttgtgagtgcggcgggccggggggcgcgggagccgttgccacgcggaccccctcggcaggagtcgggctcgcagcccgcgtgcaggccttgggcgcctttcagctctgagccttccacgttacggagccgacgcggctcccccgttagcactggggttattccttaattctaagggcggcggggcggggcggtgcttctagggctgtgtggcctgggaatggaaagggggcttcgtccggagcttgagggcgcgcgcagccgtcttgggacacaaagagtctttcccggccataagcatcgtgctcggggacgttgtctcttgctggcgcgcaaagggtgtgggggcctttcttagtgaaaagaacataaatcccacggctttctcttacccgaatcccctttttttttgtgtttgcacacgtgtttttgagggtgggtggaagggaaaggtagaagcgttgaaggaagcatgaatagttttgaactcactgggaagcaaaatcccgtcattgttccaggcactgccagggagggagcagggggcggaggttattgatccgggagagaagcgcgcgcgaagagatgctccggcctcgcgcggcggcggggatacctacgtagggaggcttttatgtgctcagccagctaatgctccctgccgctcgccctcctggctgctccgcagtcttttagatggcccgaggagcatctcgagtgtccgcgatttgggaaagagcttttaactgggctgttaaagggctgccttcttttctccaaatgcgatctggattcctgcctcggggaacagttgtgctacagagagatattttaacgccctgatattatatttcagtgtcaattttacgaattaaaaagaacaatcaaaacactagggggaaaaaaccgacaagaataacctagcaattgcataacctgagaattacttcttttcttgaacatgttcctttgagcttcttgcggaagaaattctcttaggatgagtcgtccccgacagaagatacatatttggttttctgtgtgcttcactttttggtttttcatcttggactgcacactgaatttttaaaaaacgttcatccatcaaacatttgagcacctatagggtgacggctgcctctgggcagaaggaatacgaagatccttaggaatgtaaaactcatggtgtcttcaaggcatacagaaaaaaagaagaatgtaaaactcaaatttgagataaatttttattttaagacaaactgataaatggtcaagaaaaagttttaaaatattgacataaaaaagctgtttttctcccactagattgccatgactttgtacttataaagtctactaaattatattcaaaaagtgtgtataactgtaccattttcgttaaaatattgtgtagaaaaaagtttgggggaatatataacaaataattaactgtagatccctctgggtgtaaagattacaggaggctttcactttgagcactctcaaatagtgtgaagtttggagtttttacaataagcattatgtgtgcagaaaaaattaaaatacataaacgtaaagaaacatgaaaaaatgatcagtcttaatcatgcaaattaaaactgcagtgagctatttttcctataattattgcctgagactgtacagtgaaatgttttctcatttatttatcgggtaagggtgttcaatccttttggaaagcgattttaagacatttatcagatcttaatgttcatttctgagtttggcttatatataagctttaaagggttaaaattgtatgtaggctgggtgaggtggctcatgcctgtaatcccagcactttgggaggccgaggcgggtggatcacctgaggtggggagtttgagactgcctgaccaacatggagaaaccccgtctctactaaaaatacaaaattagctgggtgtggtggtgggcgcctgtaatcccagctactcgggaggctgaggcaggagaatcatttgaaccctggaggtggaggttgcggtgggctgaaatcgcgtcactgcactccagcctgggcaacaagaatgaaactccctctcaaaaaaaaaaaattctatgtaatgtgtattacaactatgtaaaacgcatgtgtatgaagaatactggaaagaaatagagcaaaaatataagagcctttattaagtttctcaaaaggctcttctggacctctggacttgattgagtacacagtcttatttttcatagcagcctgtacctcctttatcacaatcataattaattattatttgagtaaattgttaaggtcggtaagggtagcaatcgtgtctgctgtatcttgtttagtgttctctccttcgtgcctagctcataaaaatatttagtatttgaggcccggagcagtggctcacgcctgtaatcccagcactttgggagtccccgtgggtggatcacaaggtcaggagttcaagaccagcttggccaagatggtgaagccctatctctactaaaaatacaaaattagcagggcgcggtggcaggcgcctgtaatcccagctacttgggaggctaaggctggagaattgcttcaacccaggt

SEQ ID NO: 12 - DNA, 60 bp, Artificial Sequence
  Primer
ctgcgtaaacgtcgctgtgctagctcttttgcagtcacagcttttcgtgcatgagtacgt

SEQ ID NO: 13 - DNA, 62 bp, Artificial Sequence
  Primer
agcttttcgtgcatgagtacgtattttgaaactcaagatcgcattcatgcgtcttcacgtga

SEQ ID NO: 14 - DNA, 72 bp, Artificial Sequence
  Primer
ctgcgtaaacgtcgctgtgctagctcttttgcaggcacagcttttcgtgcatgagtacgtattttgaaactc

SEQ ID NO: 15 - DNA, 55 bp, Artificial Sequence
  Primer
cgtgcatgagtacgtattttgaaactcaagatcgcattcatgcgtcttcacgtga

SEQ ID NO: 16 - DNA, 63 bp, Artificial Sequence
  Primer
ctgcgtaaacgtcgcggtgctagctcttttgcaggcacagcttttcgtgcatgagtatggatt

SEQ ID NO: 17 - DNA, 61 bp, Artificial Sequence
  Primer
gcttttcgtgcatgagtatggattttgaaactcaagatcgcattcatgcgtcttcacgtga

SEQ ID NO: 18 - DNA, 110 bp, Artificial Sequence
  Primer
ctgcttcgtgcatgagtacgtattctctttacgtgactgcgtaagtaaacgtcgctgtgctagtgcagtcacagttgaaactcaagatcgcattcaagctctttactgct

SEQ ID NO: 19 - DNA, 113 bp, Artificial Sequence
  Primer
tcgcattcaagctctttactgcttacgtgactgcgttgcagtcacagcttagtacgtattgcgtcttcagtgcatgttgaaactcaagatcgcattcatgcgtcttcacgtga

SEQ ID NO: 20 - DNA, 114 bp, Artificial Sequence
  Primer
ctgcttcgtgcatgagtacgtattctctttacgtgactgcgtaagtaaacgtcgctgtgctagtgcagtcacagttgaaactcaagatcgcattcaaggtctttactgcttacg

SEQ ID NO: 21 - DNA, 112 bp, Artificial Sequence
  Primer
cgcattcaaggtctttactgcttacgtgactgcgttgcagtcacagcttagtacgtattgcgtcttcagtgcatgttgaaactcaagatcgcattcatgcgtcttcacgtga

SEQ ID NO: 22 - DNA, 110 bp, Artificial Sequence
  Primer
ctgcttcgtgcatgcggacgtattctctttacgtgactgcgtaagtaaacgtcgctgtgctagtgcagtcacagttgaaactcaagatcgcattcaaggtcgttactgct

SEQ ID NO: 23 - DNA, 113 bp, Artificial Sequence
  Primer
tcgcattcaaggtcgttactgcttacgtgactgcgttgcagtcacagcttagtacgtattgcgtcttcagtgcatgttgaaactcaaggtcgcattcatgcgtcttcacgtga

SEQ ID NO: 24 - DNA, 50 bp, Artificial Sequence
  Primer
gccctcatgatattttaaaacacagcatcctcaaccttgaggcggaggtc

SEQ ID NO: 25 - DNA, 49 bp, Artificial Sequence
  Primer
ccttggcctggcttgcttaccttgttatcaagtcctgaatgggggaagc

SEQ ID NO: 26 - DNA, 20 bp, Artificial Sequence
  Primer
ctctcccactgcatctgtca

SEQ ID NO: 27 - DNA, 20 bp, Artificial Sequence
  Primer
catatccccatggcaaactc

SEQ ID NO: 28 - DNA, 19 bp, Artificial Sequence
  Primer
cacactgacgtgcctctcc

SEQ ID NO: 29 - DNA, 19 bp, Artificial Sequence
  Primer
gcaggtactgggagccaat

SEQ ID NO: 30 - DNA, 20 bp, Artificial Sequence
  Primer
tctccacaaggaggcatgga

SEQ ID NO: 31 - DNA, 20 bp, Artificial Sequence
  Primer
gacaggaccattgcccacag

SEQ ID NO: 32 - DNA, 27 bp, Artificial Sequence
  Primer
cgttccagaagcggcagaagcttacct

SEQ ID NO: 33 - DNA, 30 bp, Artificial Sequence
  Primer
acctgggttgaagcaattctccagccttag

SEQ ID NO: 34 - DNA, 4145 bp, Artificial Sequence
  fusion sequence
  His sequence (1)..(24)
  FLAG sequence (4122)..(4145)
catcatcatcatcatcacggatccatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccacatcgtgcctcagagctttctgaaggacgactccatcgacaacaaggtgctgaccagaagcgacaaggcccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaaggtgaaccccgacaacagctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgattacaaggatgacgatgacaag